(19) United States
(12) Patent Application Publication
(10) Pub. No.: US 2011/0017243 A1
(43) Pub. Date: Jan. 27, 2011

(54) PORTIONED PACKAGES FOR MAINTENANCE SOLUTIONS AND OPHTHALMIC LENSES

(76) Inventors: Leslie A. Voss, Jacksonville, FL (US); Catie A. Morley, St. Johns, FL (US); Dwight Abouhalkah, Jacksonville, FL (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/837,692

(22) Filed: Jul. 16, 2010

Related U.S. Application Data
(60) Provisional application No. 61/228,381, filed on Jul. 24, 2009.

(51) Int. Cl. A61L 12/00; B65D 81/00; A45C 11/04; B08B 3/04

(52) U.S. Cl. 134/42; 206/205; 206/5.1

(57) ABSTRACT

A package for a portion of maintenance solution and an ophthalmic lens and methods of using the same.

### RELATED APPLICATIONS

This application claims the benefit of provisional application, U.S. Ser. No. 61/228381 filed on Jul. 24, 2009.

### FIELD OF THE INVENTION

This invention relates to packages for ophthalmic lenses and maintenance solutions as well as methods for using the same.

### BACKGROUND

Contact lenses have been used commercially to improve vision since the 1950s. At first contact lenses were made of hard materials, such as glass and hard polymers are were packaged in glass vials. Later developments, gave rise to softer more comfortable lenses made of hydrophobic hydrogels, generally referred to as soft contact lenses. Soft contact lenses are disposable on either a daily disposable regimen or a multi-week regimen and are typically delivered to the consumer in disposable packaging commonly know as blister packages. These blister packages typically contain the ophthalmic lens and packaging solution therefore. For soft lenses with a multi-week regimen consumers remove the lenses prior to sleeping, clean the lenses and place them in a solution for storage and/or further cleaning prior to the next day's wear. Typically consumer have a container with a removable cover to house the lenses during each evening's cleaning process and a separate container that houses several more ounces of maintenance solution than are needed for a single night's use. Such containers are large bulky and most are contain too much liquid to be carried easily, or to meet the airline's travel regulations for carry-on luggage. It would be useful if there were packaging to hold the lenses during their nighttime cleaning regimen that also housed smaller quantities of maintenance solution for use with lenses. This need is met by the following invention.

### BRIEF DESCRIPTION OF THE DRAWINGS

FIGS. 1a-d illustrate a front plan view of a package the invention

FIGS. 2a-b illustrate a perspective view of a package of the invention

FIG. 3 illustrates a front plan view of a stacked package of the invention.

FIGS. 4a-c illustrate a perspective view of an interlocked package of the invention.

### DETAILED DESCRIPTION OF THE INVENTION

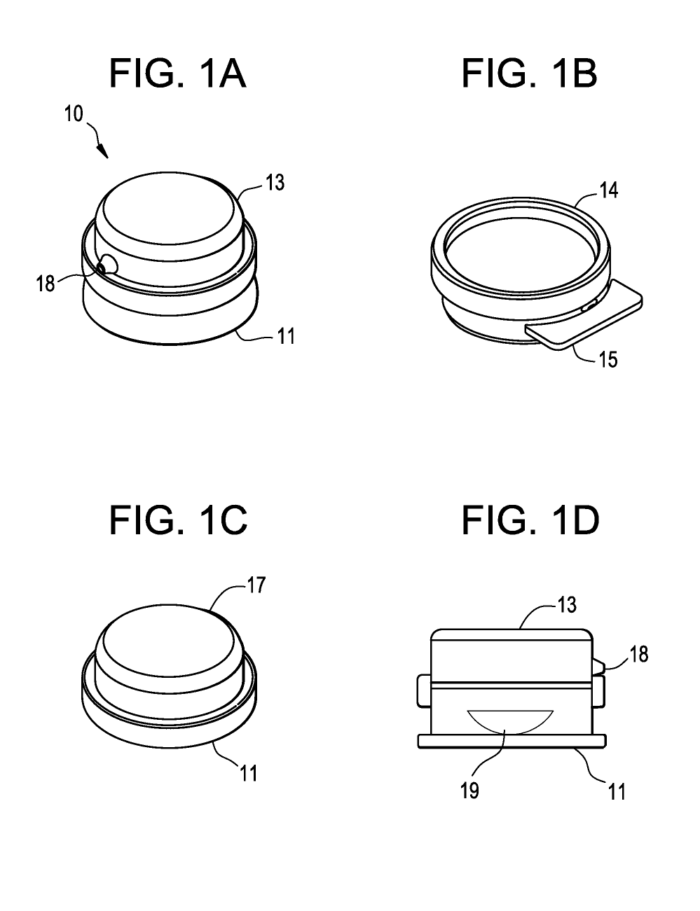

This invention includes a package for housing an ophthalmic lens and maintenance solutions therefore comprising a bowl and a cover

wherein
said bowl comprises is sized to contain an ophthalmic lens and maintenance solution and comprises an opening
said cover comprises a portion of maintenance solution and a receptacle sized to contain said of maintenance solution

wherein said cover and said bowl are removably coupled to each other. Embodiments of the invention are illustrated in FIGS. 1 through 4. FIGS. 1a-d illustrate an embodiment of the invention. Package 10 contains one bowl, 11 and cover 13. Cover 13 is a receptacle that houses maintenance solution and has a seating lip 14, and a rotatable tab 15. The cover has recess 17

As used herein “ophthalmic lens” refers to a device that resides in or on the eye. These devices can provide optical correction or may be cosmetic. Ophthalmic lenses include but are not limited to contact lenses either soft or hard. The preferred lenses of the invention are soft contact lenses.

The term “bowl” refers to the receptacle portion of an ophthalmic lens package that is sized to contain the ophthalmic lens and a portion of maintenance solution. Examples of suitably shaped bowls are disclosed in the following documents which are hereby incorporated by reference in their entirety, U.S. Pat. Nos. D 458,023; 4,691,820; 5,054,610; 5,337,888; 5,375,698; 5,409,104; 5,467,868; 5,515,964; 5,609,246; 5,695,049; 5,697,495; 5,704,468; 5,711,416; 5,722,536; 5,573,108; 5,823,327; 5,704,468; 5,983,608; 6,029,808; 6,044,966; and 6,401,915. The receptacle portion of some ophthalmic lens packages is not bowl shaped. For purposes of this invention, the receptacles of those packages are included in the term blister bowl. Examples of such packages include but are not limited to ophthalmic lens packages disclosed in WO 2005/082721, U.S. Pat. No. 7,086,526, WO 03/016175, US 2004/0238380, and US 2008/0023345 which are hereby incorporated by reference in their entirety. Unlike the bowls of the aforementioned references, the bowls of this invention are removably coupled to the covers of the invention. Preferably bowls are made of any thermoplastic material including but not limited to polypropylene, polycarbonate, or polystyrene. The bowls are sized so that a contact lens may be completely surrounded by maintenance solution. Preferably the bowl will hold about 1 mL to about 15 mL of maintenance solution, more preferably about 1 mL to about 6 mL, most preferably 2 mL to about 4 mL.

As used herein the “covers” are closed receptacles that house a portion of maintenance solution. Covers may be made of any thermoplastic material including but not limited to polypropylene, polyethylene, polycarbonate, or polystyrene. These materials can be formed in the appropriate shapes by injection molding, thermoforming, transfer molding, skin packaging, blow molding, coinjection molding, film extrusion, blow-fill-seal manufacturing or film coextrusion and the like. Preferably covers are made from polyethylene and by blow-fill-seal manufacturing.

The covers hold a “portion” of maintenance solution typically about 2 mL to about 30 mL of maintenance solution, more preferably about 2 mL to about 15 mL, most preferably about 2 mL to about 10 mL. The preferably covers of the invention house enough maintenance solution to clean a single contact lens once. In the embodiment of FIG. 1, covers 13a and 13b have removable tabs to close apertures 18a and 18b and it is preferred to have removable tabs on the covers but not required. Other devices may be used to release the maintenance solution in the cover such as a piercing mechanism formed on either the bowl or the cover. Further in this embodiment, covers 13a and 13b are sealed in order to prevent contamination of the maintenance solution contained therein and it is preferred that the covers of the invention are sealed.

The term “maintenance solution” refers to a liquid medium any composition which can be directly instilled into an eye, or which can be used to soak, clean, rinse, store or treat any ophthalmic device which can be used placed in or on the eye. Examples of maintenance solutions include ophthalmic device packing solutions, cleaning solutions, conditioning solutions, storage solutions, eye drops, eye washes, as well as suspensions, gels and ointments and the like. Examples of such solutions include those disclosed in the following publications U.S. patent application Ser. No. 12/124,882, entitled “STABILIZED OPHTHALMIC SOLUTIONS” and U.S. patent application Ser. No. 12/399,662, entitled OPHTHALMIC SOLUTIONS DISPLAYING IMPROVED EFFICACY, which are hereby incorporated by reference in their entirety.

In order to use the package of FIGS. 1a-d during cleaning, the user rotates removable tab stop 15 to reveal aperture 18. A few drops of solution contained in cover 13 may be used to initially rinse ophthalmic lens, 19, then apertures 18 is oriented towards recess 17 to dispense the remainder of the maintenance solution. Cover 13 closes recesses 17 via seating lip 14 that recess to leave the ophthalmic lens in the maintenance solution.

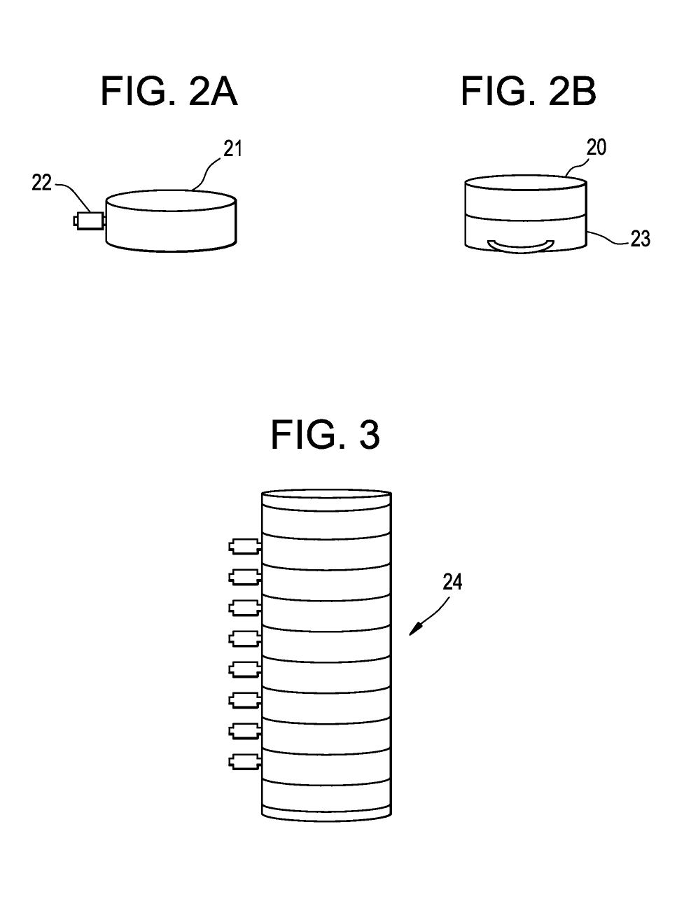

Another embodiments of the invention are illustrated in FIGS. 2 and 3. Package 20 has cover 21 of FIG. 2, has removable tabs 22 which cover aperture (not shown) Cover 21 fits over bowl 23 to contain an ophthalmic lens and its maintenance solution therein. In FIG. 3, more than two bowls 23 are pressure fitted against a stack of covers 21 that nest to each other as well as to bowls 23. This stacked package 24 holds several days of maintenance solutions

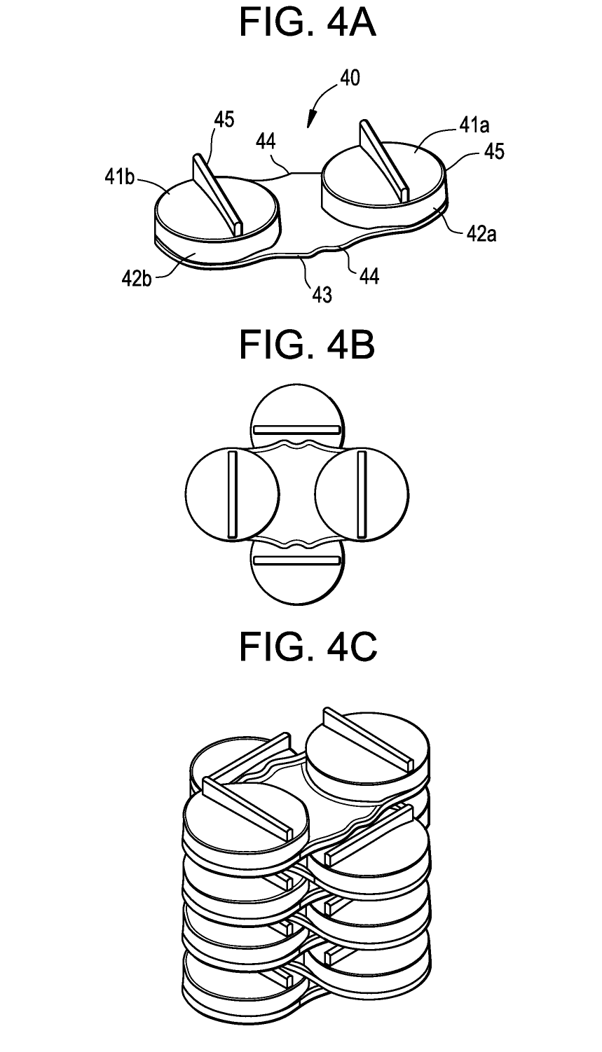

FIGS. 4a-c illustrate package 40 and a stack of the same packages interlocked to one another. Covers 41a and 41b rest within bowls 42a and 42b. Tab 43 connects bowls 42a and 42b to each other and the tab contains two locking ridges 44. Tabs 45a and 45b may be snapped off to reveal apertures (not illustrated) for dispensing of the maintenance solution. FIG. 4billustrates two packages stacked at 90 degree angles and locked together at the intersection of removable tabs 45 with locking ridges 44. FIG. 4c illustrates several packages stacked together.

Further the invention includes a method of cleaning an ophthalmic lens comprising
placing an ophthalmic lens in a package comprising a bowl and a cover

wherein
said bowl comprises is sized to contain an ophthalmic lens and maintenance solution and comprises an opening
said cover comprises a portion of maintenance solution and a receptacle sized to contain said of maintenance solution

wherein said cover and said bowl are removably coupled to each other.
depositing maintenance solution from said cover into said bowl and placing the ophthalmic lens in said bowl.
   As used herein the terms bowl, cover, maintenance solution and portion all have their aforementioned meanings and preferred ranges.

What is claimed is:

1. A package for housing an ophthalmic lens and maintenance solutions therefore comprising a bowl and a cover
wherein
said bowl comprises is sized to contain an ophthalmic lens and maintenance solution and comprises an opening
said cover comprises a portion of maintenance solution and a receptacle sized to contain said of maintenance solution
wherein said cover and said bowl are removably coupled to each other.

2. The package of claim 1 wherein said cover further comprises removable tab which seals said maintenance solution in said cover.

3. The package of claim 1 wherein said portion of maintenance solution is about 2 mL to about 30 mL.

4. The package of claim 1 wherein said cover is made from a material comprising one or more members of group consisting of polypropylene, polyethylene, polycarbonate, or polystyrene

5. The package of claim 1 wherein said bowl is made from a material comprising one or more members of group consisting of polypropylene, polyethylene, polycarbonate, and polystyrene.

6. A method of cleaning an ophthalmic lens comprising
placing an ophthalmic lens in a package comprising a bowl and a cover
wherein
said bowl comprises is sized to contain an ophthalmic lens and maintenance solution and comprises an opening
said cover comprises a portion of maintenance solution and a receptacle sized to contain said of maintenance solution
wherein said cover and said bowl are removably coupled to each other.
depositing maintenance solution from said cover into said bowl and
placing the ophthalmic lens in said bowl.

7. The method of claim 6 wherein said cover further comprises removable tab which seals said maintenance solution in said cover.

8. The method of claim 6 wherein said portion of maintenance solution is about 2 mL to about 30 mL.

9. The method of claim 6 wherein said cover is made from a material comprising one or more members of group consisting of polypropylene, polyethylene, polycarbonate, and polystyrene.

10. The method of claim 6 wherein said bowl is made from a material comprising one or more members of group consisting of polypropylene, polyethylene, polycarbonate, and polystyrene.
